(19) United States
(12) Patent Application Publication
(10) Pub. No.: US 2018/0099116 A1
(43) Pub. Date: Apr. 12, 2018

(54) SYSTEM FOR MONITORING AND CONTROLLING SLEEP

(71) Applicant: ICON Health & Fitness, Inc., Logan, UT (US)

(72) Inventor: Darren C. Ashby, Richmond, UT (US)

(73) Assignee: ICON Health & Fitness, Inc., Logan, UT (US)

(21) Appl. No.: 15/836,650

(22) Filed: Dec. 8, 2017

Related U.S. Application Data
(63) Continuation-in-part of application No. 14/932,455, filed on Nov. 4, 2015.
(60) Provisional application No. 62/075,744, filed on Nov. 5, 2014.

(51) Int. Cl. A61M 21/02

(52) U.S. Cl. CPC A61M 21/02; A61M 2021/0022

(57) ABSTRACT

A system for monitoring and controlling a heart rate includes an interface for communicating with a heart rate monitor and a haptic device. The system also includes a processor and memory. The memory includes programmed instructions to cause the processor to determine a natural heart rate of a user, determine a target rate for the user, and cause the haptic device to provide haptic input to the user to adjust the natural heart rate to the target heart rate.

### RELATED APPLICATIONS

This application is a continuation-in-part of U.S. patent application Ser. No. 14/932,455 filed on 4 Nov. 2015 and titled “System with a Heart Rate Adjusting Mechanism,” now published as U.S. Patent Publication No. 2016/0121074; which application claims priority to U.S. Patent Application Ser. No. 62/075,744 filed on 5 Nov. 2014 and titled “System with a Heart Rate Adjusting Mechanism”; which applications are herein incorporated by reference for all that they disclose.

### BACKGROUND

Sleep provides many benefits to humans and animals. While there is still much to learn about sleep, research suggests that during sleep restorative functions occur in the nervous, skeletal, and muscular systems. Also, memory loss has been associated with sleep deprivation suggesting that sleep plays a role in retaining memory. Some experts believe that people should get at least six hours of sleep a night. However, due to sleep disorders, some find getting adequate sleep difficult.

One type of device for determining whether a person is asleep is disclosed in U.S. Pat. No. 5,479,939 issued to Hiroyuki Ogino. In this reference, movement of a person in bed is detected without contacting the body, and time measurement is reset and started newly by a timer every time a detected movement exceeds a predetermined set value. When the measurement time of the timer exceeds a set time predetermined, it is judged that the body has fallen asleep on the bed. Meanwhile, absence or presence in bed and rough body movement are judged by detecting the fine body movement propagated by the functioning of heart and breathing of the body. Another type of system is described in U.S. Patent Publication No. 2009/0178199 issued to Andreas Brauers, et al. Each of these documents are herein incorporated by reference for all that they contain.

While these and other such systems typically monitor the sleep patents of a person, they do not actively aid in providing conditions to allow a person to fall asleep quicker or experience more restful sleep. The data collected by these systems may be useful in diagnosing sleep problems, however a doctor or other such medical professional may still need to provide solutions to these problems, for example via drugs or other methods.

### SUMMARY

In one embodiment, a system for adjusting a heart rate may include an interface in communication with a heart rate monitor and a haptic device, and a processor and memory, the memory including programmed instructions to cause the processor to determine a natural heart rate of a user, determine a target heart rate for the user, and cause the haptic device to provide a haptic input to the user to adjust the natural heart rate to the target heart rate.

The system may further include the heart rate monitor in communication with the interface.

The system may further include the haptic device in communication with the interface.

The haptic device may include a cone-less speaker.

The haptic device may include an electromagnetic transducer.

The haptic device may be incorporated into a bed, an article of clothing, a wearable device, or combinations thereof.

The target heart rate may be configured to assist the user with waking out of a slumber.

The target heart rate may be higher than the natural heart rate.

The haptic input may be adjusted over a time period, and the haptic input at a beginning of the time period may be closer to the natural heart rate and the haptic input at an end of the time period may be closer to the target heart rate.

The haptic input may change incrementally during the time period. The time period may end when the natural heart rate reaches the target heart rate, and the programmed instructions cause the haptic device not to provide haptic input while the natural heart rate is within a predetermined amount of the target heart rate.

The haptic input at a beginning of the time period may be closer to the natural heart rate and the haptic input at an end of the time period is closer to the target heart rate.

The programmed instructions may further cause the processor to determine if the natural heart rate is within a predetermined amount of the target heart rate, cause the haptic device to not provide haptic input when the natural heart rate is within the predetermined amount of the target heart rate, and cause the haptic device to provide the haptic input to the user to adjust the natural heart rate to the target heart rate when the natural heart rate is not within the predetermined amount of the target heart rate.

The interface may be for further communicating with a second heart rate monitor, wherein the programmed instructions further cause the processor to determine the natural heart rate of a second user, determine the target heart rate of the second user, and cause the haptic device to provide a second haptic input to the second user to adjust the natural heart rate to the target heart rate of the second user, wherein the haptic input and the second haptic input are provided independently from one another.

According to some embodiments, a system for adjusting a heart rate may include a communication interface, a heart monitor in communication with the communication interface, a haptic device in communication with the communication interface, and a processor and memory, the memory including programmed instructions to cause the processor to determine a natural heart rate of a user, determine a target heart rate configured to assist the user with sleeping, and cause the haptic device to provide a haptic input to the user to adjust the natural heart rate to the target heart rate where the haptic input is adjusted over a time period such that the haptic input at a beginning of the time period is closer to the natural heart rate and the haptic input at an end of the time period is closer to the target heart rate.

The haptic input may be changed incrementally during the time period.

The time period may end when the natural heart rate reaches the target heart rate.

The programmed instructions may cause the haptic device to cease providing haptic input while the natural heart rate is within a predetermined amount of the target heart rate.

The programmed instructions may cause the processor to determine if the natural heart rate is within a predetermined amount of the target heart rate, cause the haptic device to cease providing haptic input when the natural heart rate is within the predetermined amount of the target heart rate, and cause the haptic device to provide the haptic input to the user to adjust the natural heart rate to the target heart rate when the natural heart rate is outside the predetermined amount of the target heart rate.

The interface may communicate with a second heart rate monitor and the programmed instructions further cause the processor to determine the natural heart rate of a second user, and determine the target heart rate of the second user, cause the haptic device to provide a second haptic input to the second user to adjust the natural heart rate to the target heart rate of the second user where the haptic input and the second haptic input are provided independently from one another.

The heart monitor may be worn on a body of the user. The haptic device may be incorporated into a bed, an article of clothing, a wearable device, or combinations thereof. The heart monitor may be in wireless communication with the processor. The target heart rate may be determined based on a desired state of a sleep or wakefulness of the user.

In one embodiment, a system for adjusting a heart rate may include a communication interface, a heart monitor worn by a user and in communication with the communication interface, a haptic device incorporated into a bed, an article of clothing, or a wearable device in communication with the communication interface, and a processor and memory, the memory including programmed instructions to cause the processor to determine a natural heart rate of a user, determine a target heart rate configured to assist the user with sleeping or waking, cause the haptic device to provide a haptic input to adjust the natural heart rate to the target heart rate where the haptic input is adjusted incrementally over a time period such that the haptic input at a beginning of the time period is closer to the natural heart rate and the haptic input at an end of the time period is closer to the target heart rate, and cause the haptic device to not provide haptic input when the natural heart rate is within a predetermined amount of the target heart rate.

### BRIEF DESCRIPTION OF THE DRAWINGS

The accompanying drawings illustrate various embodiments of the present apparatus and are a part of the specification. The illustrated embodiments are merely examples of the present apparatus and do not limit the scope thereof.

FIG. 1 illustrates a perspective view of an example of a system incorporated into a bed in accordance with the present disclosure.

FIG. 2 illustrates a top view of an example of a heart rate monitor incorporated into a bed in accordance with the present disclosure.

FIG. 3 illustrates a top view of an example of a haptic device in accordance with the present disclosure

FIG. 4 illustrates a diagram of an example of changing a natural heart rate in accordance with the present disclosure.

FIG. 5 illustrates a block diagram of an example of an adjustment system in accordance with the present disclosure.

FIG. 6 illustrates a block diagram of an example of a method for adjusting a natural heart rate in accordance with the present disclosure.

FIG. 7 illustrates an example of a haptic device incorporated into an article of clothing in accordance with the present disclosure

Throughout the drawings, identical reference numbers designate similar, but not necessarily identical, elements.

### DETAILED DESCRIPTION

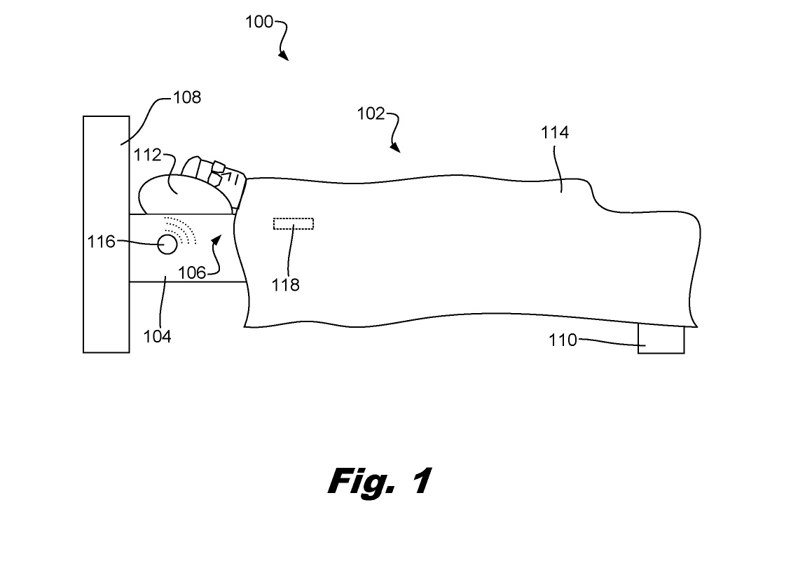

FIG. 1 depicts a perspective view of an example a system 100 for monitoring and controlling sleep incorporated into a bed 102. In this example, the bed 102 includes a bed frame 104, a mattress 106, a head board 108, bed legs 110, a pillow 112, and a blanket 114. When a user desires to sleep, the user can lay down on the mattress 106 and rest his or her head on the pillow 112. Depending on the temperature in the room, the user can desire to pull the blanket 114 over himself or herself.

Some users can experience sleep disorders where it is difficult for a user to fall asleep or stay asleep. To assist these and other users, the adjustment system 100 can detect the user's heartbeat with a heart rate monitor 118. Any appropriate type of heart rate monitor can be used in accordance with the principles described in the present disclosure. For example, the user can wear a heart rate monitor that is in communication with a processor of the adjustment system 100.

The adjustment system 100 can provide haptic input to a user, for example through a haptic device 116 that mimics the target heart rate to be directed towards the user. In some examples, a haptic device 116 is incorporated into the bed 102, and the processor can cause the haptic device 116 to provide haptic input to the user. In such an example, the haptic input can have the effect of causing the user's heart rate to slow down or speed up to the same level as the target heart rate. By bringing down the user's heart rate, the user can start to advance into the early stages of the sleeping cycle. Thus, the haptic input can assist the user with falling asleep. In some cases, by bringing up the user's heart rate, the user can start to advance from a state of sleep or slumber to a state of wakefulness. Thus, the haptic input can assist the user with waking form sleep.

In the illustrated example, the haptic device 116 is incorporated into the bed frame 104. In such an example, the haptic input can be directed to the user through direct contact with the user and/or through the bed frame 104, mattress 106, or other materials that make up the bed 102. However, in other examples, the haptic device 116 can be incorporated into an article of clothing or wearable device worn by the user during sleep. In these cases, the haptic device 116 can provide haptic input to the user via contact with the user.

In some examples, the haptic input provided by the haptic device 116 is directed to the user primarily through vibrations in the media of the bed, and the user primarily picks up the haptic input through his or her tactile senses. However, any appropriate mechanism for directing the haptic input to the user can be used in accordance with the principles described herein.

The adjustment system 100 can automatically turn on in response to detecting a heartbeat through the heart rate monitor 118. In other examples, the adjustment system 100 automatically activates in response to detecting a heart rate when the lights are out in the room with the bed 102. In yet other examples, the adjustment system 100 automatically activates in response to detecting a heart rate during certain time periods, such as the night time or evening. In other examples, the detecting of a heart rate is not used to activate the adjustment system 100. In such cases, the time of day, identification of the user through a camera, detection of a person on the bed through weight sensors or a wireless proximity device, other mechanisms for detecting that conditions are right to activate the adjustment system 100, or combinations thereof can be used to active the adjustment system 100.

In yet other cases, the adjustment system 100 can activate in response to the user providing a command to the system to activate. For example, as the user lies down to sleep, the user can instruct the adjustment system 100 to turn on by flipping a switch, pressing a button, touching a touch screen input, sending a message through a mobile device, providing a speech command, providing instruction through another type of input mechanism, or combinations thereof.

In examples where the user wears his or her own heart rate monitor 118, the adjustment system 100 can determine the identity of the user based on an identifier of the heart rate monitor. In one example, the personal heart rate monitor 118 can include an identification code in a signal that contains the heart rate information sent to the processor. In other examples, a camera can be located in the room with the bed or on a personal computing device such as a mobile phone, and the adjustment system 100 can identify the identity of the user through the camera.

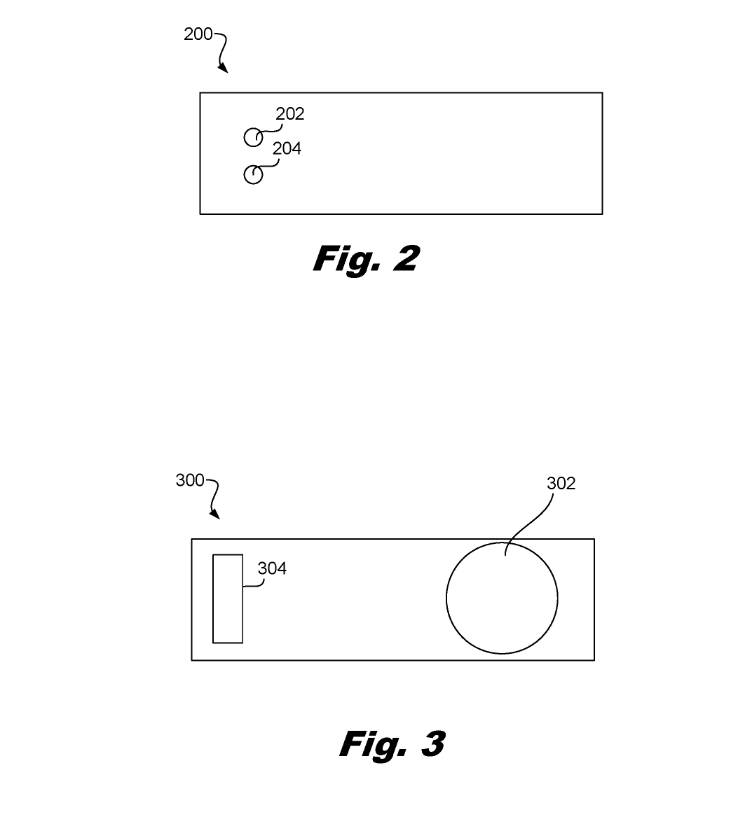

FIG. 2 illustrates a top view of an example of a heart rate monitor 200 incorporated into a bed 102 in accordance with the present disclosure. In this example, a first electrode 202 and a second electrode 204 are incorporated into a mattress 106 of the bed 102. These electrodes 202, 204 can be used to detect a voltage that represents the user's heart rate. As the user lies down, the electrodes 202, 204 can come into contact with the user's skin such that the electrodes 202, 204 can detect electrocardiography (ECG) signals of the user.

The electrodes 202, 204 can be solid metal pieces that come into contact with any appropriate parts of the user's body when the user lies down on the mattress 106. In some examples, the electrodes 202, 204 are positioned to come into contact with the user's arms, chest, legs, neck, feet, wrists, upper body, lower body, other portions of the user's body, or combinations thereof.

In other examples, the electrodes 202, 204 come into contact with the user's skin indirectly. In such examples, the electrodes 202, 204 can be buried beneath the surface of the mattress 106, but the electrodes 202, 204 come into direct contact with an electrically conductive portion of the surface of the mattress 106. Such electrically conductive portions of the mattress 106 can be flexible to provide the user with more comfort as he or she lies down on the mattress 106. In some examples, the sheets on the mattress 106 and/or the user's clothing have electrically conductive portions of fabric that come into direct contact with either the electrodes 202, 204 or electrically conductive portions of the mattress 106. Thus, while the electrodes 202, 204 may not come into direct contact with the user's skin, an electrically conductive pathway can be formed between the electrodes 202, 204 and the user's skin such that the electrodes 202, 204 can detect the user's heart rate.

In other examples, the principles described above in relation to the electrodes 202, 204 incorporated into the mattress 106 can be applied to electrodes incorporated into other portions of the bed. For example, the electrodes 202, 204 can be incorporated into the bed frame 104, the pillow 112, the blanket 114, another portion of the bed 102, or combinations thereof.

While the examples above have been described with reference to heart rate monitors that use electrical contact to determine the user's heart rate, other types of heart rate monitors can be used in accordance with the principles described herein. For example, the inductive and capacitive mechanisms for determining the user's heart rate can be used in accordance with the principles described herein. Further, the heart rate monitor or monitors need not be incorporated into the bed, and can be incorporated into, for example, an article of clothing worn by the user, a wearable device worn by the user such as a watch, or some combination thereof. In some examples, a user's heart rate can be determined without contacting the user, for example, by a camera that can detect the user's heart rate through changes in the color of the user's skin and/or other methods.

FIG. 3 illustrates a top view of an example haptic device 300 in accordance with the present disclosure. In this example, a haptic actuator 302 provides haptic input to a user as described herein. In some examples, the haptic actuator 302 can provide haptic input to a user through contact with the user, however in other example the haptic actuator can provide haptic input to the user through a medium, such as a bed or through the air with waves. The haptic input can be provided to the user in the form of vibrations generated by the haptic actuator 302. In some cases, the haptic actuator 302 can cause vibrations to travel through the medium to the user, as described herein.

In some examples, the haptic actuator 302 can be electromagnetically actuated and can be, for example, an electromagnetic transducer. That is, in some examples, the haptic actuator 302 can include a wire coil and a magnet disposed there through. An electrical current can be run through the wire coil, for example at a desired frequency, in order to cause the magnetic to move. The magnet can in turn be attached to further components to provide this vibration to the user. In some examples, the haptic actuator can be an electromagnetic transducer. In some examples, the haptic actuator 302 can be a cone-less speaker. the In some other examples, the haptic actuator 302 can be an eccentric rotating mass (ERM) actuator and can include an unbalanced weight attached to a motor shaft. As the shaft rotates, the spinning of the irregular mass causes the actuator, and in turn, the haptic device 302, to shake or vibrate. In some embodiments, other methods and devices can be used to provide haptic input and the haptic actuator 302 can include a combination of the technology described above.

In one examples, the cone-less speaker can include a device that can be mounted to a variety of surfaces, such as glass, clothing, bed frames, drywall, and so forth. These speakers can generally use a low frequency that can propagate the sound through the material. These speakers can be used to transmit a haptic input to the user through the user's bed. An example of a cone-less speaker that can be compatible with the present disclosure can be purchased through Mad Systems, Inc. with an office at 733 North Main Street, Orange, Calif. 92868, U.S.A.

In some embodiments, the haptic device 300 can also include an antenna 304 for wireless communication with the processor of an adjustment system as described herein. In some examples, the wireless antenna 304 can provide for communication via Bluetooth, Wi-Fi, radio waves, or any form of wireless communication now known or as can be developed in the future.

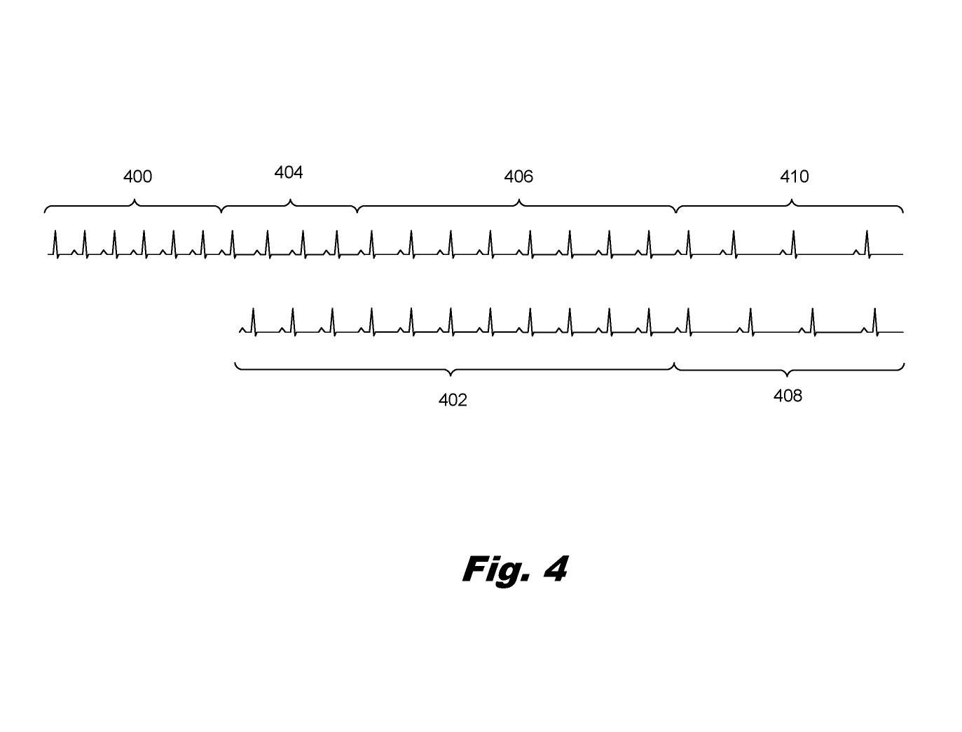

FIG. 4 illustrates a diagram of an example of changing a natural heart rate 400 in accordance with the present disclosure. In this example, the natural heart rate 400 is depicted as having a specific rate. As time passes, a first haptic input 402 is provided to the user. The first haptic input has a slower beat than the natural heart rate 402. As the user perceives the first haptic input 402, the user's body causes the user's heart rate to mimic the beat of the first haptic input 402. Thus, the user's heart rate changes during a first transition phase 404. At the end of the first transition time 404, the user's current heart rate 406 has the same rate as the first haptic input 402. In some cases, the user's perception of the haptic input 402 may not be a conscious perception. Thus, while the haptic input 402 can cause a physiological response in the user, the user can not necessarily be consciously aware of the haptic input 402 or the physiological response.

In FIG. 4, the adjustment system 100 changes the user's natural heart rate to the target heart rate through multiple incremental haptic inputs with progressively slower beats. In the illustrated example, a second sound 408 is directed towards the user after the user's current heart rate 408 mimics the first haptic input 402. The second haptic input 408 can be closer to the target heart rate than the first haptic input 402. As a result, the user's current heart rate 406 enters into a second transition phase 410. During the second transition phase 410, the user's current heart rate 406 slows down to mimic the beat rate of the second haptic input 408.

This incremental process can repeat itself until the user's heart rate mimics the target heart rate with each of the haptic inputs directed towards the user during each time increment. During each time increment, the haptic inputs can have beat rates that progressively get closer to the target heart rate.

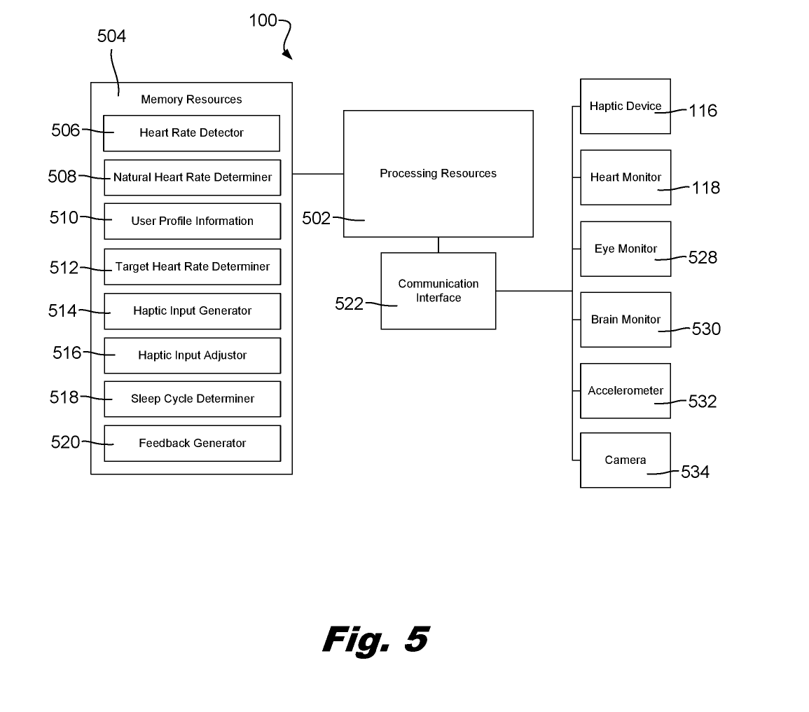

FIG. 5 illustrates a perspective view of an example of an adjustment system 100 in accordance with the present disclosure. The adjustment system 100 can include a combination of hardware and programmed instructions for executing the functions of the adjustment system 100. In this example, the adjustment system 100 includes processing resources 502 that are in communication with memory resources 504. Processing resources 502 include at least one processor and other resources used to process the programmed instructions. The memory resources 504 represent generally any memory capable of storing data such as programmed instructions or data structures used by the adjustment system 100. The programmed instructions and data structures shown stored in the memory resources 504 include a heart rate detector 506, a natural heart rate determiner 508, user profile information 510, a target heart rate determiner 512, a haptic input generator 514, a haptic input adjustor 516, a sleep cycle determiner 518, and a feedback generator 520.

The processing resources 502 can be in communication with communications interface 522 that communicates with external devices. Such external devices can include a haptic device 116, a heart rate monitor 118, an eye monitor 528, a brain monitor 530, an accelerometer 532, a camera 534, another external device, or combinations thereof. In some examples, the processing resources 502 communicate with the external devices through a mobile device which wirelessly relays communications between the processing resources 502 and the remote devices.

The external devices can gather information or execute a task to carry out a purpose of the adjustment system 100. For example, a haptic device 116 can provide haptic input to the user in response to receiving a command from the processing resources 502. Further, the heart rate monitor 118 can collect information about the user's natural heart rate or at least the user's current heart rate, which can be used by the processing resources to determine which sounds to direct towards the user. The eye monitor 528 can be used to detect eye movement to assist the adjustment system 100 in determining whether the user is currently experiencing non-REM sleep or REM sleep.

The brain monitor 530 can be an electroencephalogram, a magnetoencephalogram, another type of brain monitor, or combinations thereof that can pick up waveforms generated by brain activity. As neurons in the brain fire, they create electrical signals that can be detected. During different stages of sleep, the brain's activity produces different types of patterns. For example, alpha waves usually have a frequency of 8.0 to 15.0 and are often exhibited during the first stage of non-REM and during REM sleep. Theta waves often exhibit a frequency of 4.0 to 7.0 hertz and are often exhibited during the second stage of non-REM sleep and REM sleep. A delta wave usually has a frequency of 1.0 to 4.0 hertz and is often exhibited during a third stage of sleep. During REM sleep, the user's brain activity often appears to be similar to when the user is awake. Thus, the brain monitor 530 can be used to determine the sleep cycle that the user is currently experiencing. As a result, the adjustment system 100 can tailor the target heart rate to be appropriate to the particular sleep stage being experienced by the user.

The accelerometer 532 can be used to determine whether the user is moving in his or her sleep. Such information can assist the adjustment system 100 in determining whether the user is in a deep sleep, REM sleep, an initial cycle of sleep, and so forth. Such information can be used to determine the appropriate target heart rate for the user based in part on the user's current sleep stage. A camera 534 can also be used to determine the user's body motions and/or restlessness and in some cases can be used to determine a user's heart rate.

Further, the communication interface can be in communication with a database that contains information about the user. An example of a database that can be compatible with the principles described herein includes the iFit program as described above. In some examples, the user information accessible through the communication interface includes the user's age, gender, body composition, height, weight, health conditions, other types of information, or combinations thereof that can be helpful in determining the appropriate target heart rate for the user.

The processing resources 502, memory resources 504 and external devices can communicate over any appropriate network and/or protocol through the communications interface 522. In some examples, the communications interface 522 includes a transceiver for wired and/or wireless communications. For example, these devices can be capable of communicating using the ZigBee protocol, Z-Wave protocol, BlueTooth protocol, Wi-Fi protocol, Global System for Mobile Communications (GSM) standard, another standard or combinations thereof. In other examples, the user can directly input some information into the adjustment system 100 through a digital input/output mechanism, a mechanical input/output mechanism, another type of mechanism or combinations thereof.

The memory resources 504 include a computer readable storage medium that contains computer readable program code to cause tasks to be executed by the processing resources 502. The computer readable storage medium can be a tangible and/or non-transitory storage medium. The computer readable storage medium can be any appropriate storage medium that is not a transmission storage medium. A non-exhaustive list of computer readable storage medium types includes non-volatile memory, volatile memory, random access memory, write only memory, flash memory, electrically erasable program read only memory, magnetic based memory, other types of memory or combinations thereof.

The heart rate detector 506 represents programmed instructions that, when executed, cause the processing resources 502 to detect the heart rate of the user. This can be accomplished in response to the heart rate monitor 118 sending information to the processing resources 502. The natural heart rate determiner 508 represents programmed instructions that, when executed, cause the processing resources 502 to determine the natural heart rate of the user. Such a determination can be based on the information from the heart rate monitor 118.

The user profile information 510 can be stored in the memory resources 504 or in a database in communication with the processing resources 502 through the communications interface 522. Such user information can include data about the user's age, gender, health conditions, weight, body compositions, and so forth that can be used to determine the target heart rate for the user.

The target heart rate determiner 512 represents programmed instructions that, when executed, cause the processing resources 502 to determine the target heart rate. In some examples, known target heart rates that can be used for a wide variety of people to assist them with sleeping or waking are used. In such an example, little personal data, if any, can be necessary to assist the user with sleeping or waking. In other examples, the target heart rate is determined based on just the natural heart rate of the user. In such examples, the target heart rate determiner 512 can use an equation to determine the target heart rate. In some cases, the equation can be a percentage of the user's resting heart rate. For example, if the user is resting on the bed 102 and the natural resting heart rate of the user is 75 beats per minute, and the equation is

0.9(resting heart rate)=target heart rate,

than the target heart rate can be determined to be 67.5 beats per minute. While this example has been described with a specific equation, any equation, procedure, or other mechanism for determining the user's target heart rate can be used in accordance with the principles described above.

The haptic input generator 514 represents programmed instructions that, when executed, cause the processing resources 502 to generate haptic input that has a beat rate that is at least substantially similar to the target heart rate or at least a predetermined incremental beat customized to assist the user's heart rate to slowly adjust to the target heart rate. For example, the haptic input generator can cause a first haptic input to be provided to the user that is slower than the user's current heart rate, but faster than the target heart rate.

The haptic input adjustor 516 represents programmed instructions that, when executed, cause the processing resources 502 to adjust the haptic input as appropriate. For instance, if the haptic input provided to the user does not represent the target heart rate, the haptic input adjustor 516 causes the haptic input to be adjusted such that the haptic input progressively get closer to the target heart rate. Likewise, as the user progresses through the sleep stages and/or sleep cycles, the target heart rate can change, and the haptic input adjustor 516 can cause haptic inputs to change accordingly. In some cases, the haptic input adjustor can represent programmed instructions which cause the haptic device to not provide haptic input when the natural heart rate of the user is within the predetermined amount of the target heart rate. In this case, the programmed instructions can further cause the haptic device to provide the haptic input to the user to adjust the natural heart rate to the target heart rate when the natural heart rate is not within the predetermined amount of the target heart rate.

The sleep cycle determiner 518 represents programmed instructions that, when executed, cause the processing resources 502 to determine the sleep stage and/or sleep cycle of the user. This information can be used by the target heart rate determiner 512 to determine an appropriate target heart rate.

The feedback generator 520 represents programmed instructions that, when executed, cause the processing resources 502 to generate feedback to determine the effectiveness of the target heart rate. For example, if the haptic input generated by the adjustment system 100 causes the user to fall asleep quickly, the feedback generator 520 can determine that the generated haptic input was effective. However, if the haptic input causes the user to have delayed sleep, to undesirably wake up, or to take longer than desired to fall asleep, the feedback generator can adjust the target heart rate and/or the intermediary haptic input used to help the user's heart rate arrive at the target heart rate. In some examples, the beats of the intermediary haptic input can be adjusted. In other examples, the increment times where the intermediary haptic input is produced can be adjusted by the feedback generator to increase the effectiveness of the adjustment system 100. Thus, the adjustment system 100 can include one or more learning algorithms for increasing the effectiveness of helping the user to sleep or wake.

Further, the memory resources 504 can be part of an installation package. In response to installing the installation package, the programmed instructions of the memory resources 504 can be downloaded from the installation package's source, such as a portable medium, a server, a remote network location, another location or combinations thereof. Portable memory media that are compatible with the principles described herein include DVDs, CDs, flash memory, portable disks, magnetic disks, optical disks, other forms of portable memory or combinations thereof. In other examples, the program instructions are already installed. Here, the memory resources 504 can include integrated memory such as a hard drive, a solid state hard drive or the like.

In some examples, the processing resources 502 and the memory resources 504 are located within the heart rate monitor 118, the haptic device 116, the bed 102, a component of the bed 102, the user's clothing, a mobile device, an external device, another type of device, or combinations thereof. The memory resources 504 can be part of any of these device's main memory, caches, registers, non-volatile memory, or elsewhere in their memory hierarchy. Alternatively, the memory resources 504 can be in communication with the processing resources 502 over a network. Further, data structures, such as libraries or databases containing user, can be accessed from a remote location over a network connection while the programmed instructions are located locally. Thus, the adjustment system 100 can be implemented with the mobile device, an external device, a phone, an electronic tablet, a wearable computing device, a head mounted device, a server, a collection of servers, a networked device, a watch, or combinations thereof. Such an implementation can occur through input/output mechanisms, such as push buttons, touch screen buttons, speech commands, dials, levers, other types of input/output mechanisms, or combinations thereof. Any appropriate type of wearable device can include, but are not limited to glasses, arm bands, leg bands, torso bands, head bands, chest straps, wrist watches, belts, earrings, nose rings, other types of rings, necklaces, garment integrated devices, other types of devices, or combinations thereof.

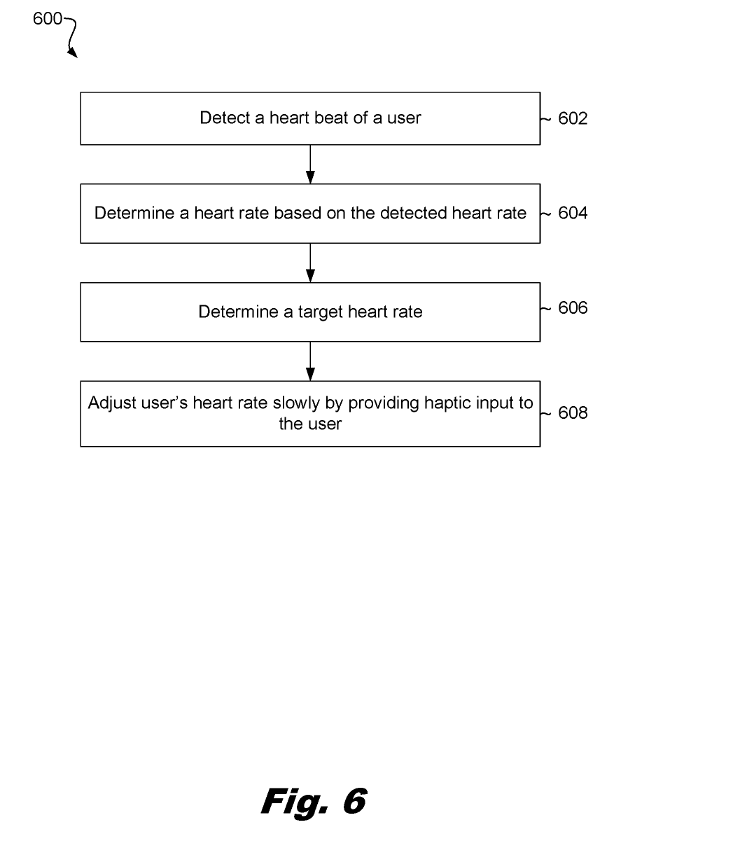

FIG. 6 illustrates a block diagram of an example of a method 600 for adjusting a natural heart rate in accordance with the present disclosure. In this example, the method 600 includes detecting 602 a heartbeat of a user, determining 604 a heart rate based on the detected heart rate, determining 606 a target heart rate, and adjusting 608 the user's heart rate slowly by directing haptic input towards the user.

At block 602, the heartbeat is detected. Such a heartbeat can be detected by the heart rate monitor or another type of device. At block 604, the natural heart rate is determined based at least in part on the detected heartbeat. In some examples, the heart rate is determined by counting the number of beats detected within a predetermined time period. In other examples, the signals from the heart rate monitor are filtered to remove noise or other distortions in the signal.

At block 606, the target heart rate is determined. The target heart rate can be based on applying an equation to the user's heart rate. In other examples, personal information about the user is also used to determine the target heart rate. For example, the user's age, gender, health, weight, body composition, and so forth can be used to determine the target heart rate. In some cases the target heart rate can be slower than the detected heart rate, for example when a user is attempting to sleep. However, in other cases the target heart rate can be faster than the detected heart rate, for example when a user is being woken up.

At block 608, the user's heart rate is adjusted, for example slowed or sped up, by directing haptic input to the user. Such haptic input can have a beat that is at least similar to the target heart rate. In some examples, the haptic input has a beat that is slower than the user's current heart rate, but faster than the target heart rate. In other examples, the haptic input can have a beat that is faster than the user's current heart rate, but slower than the target heart rate. The user's heart rate can be adjusted in incremental stages or all at once.

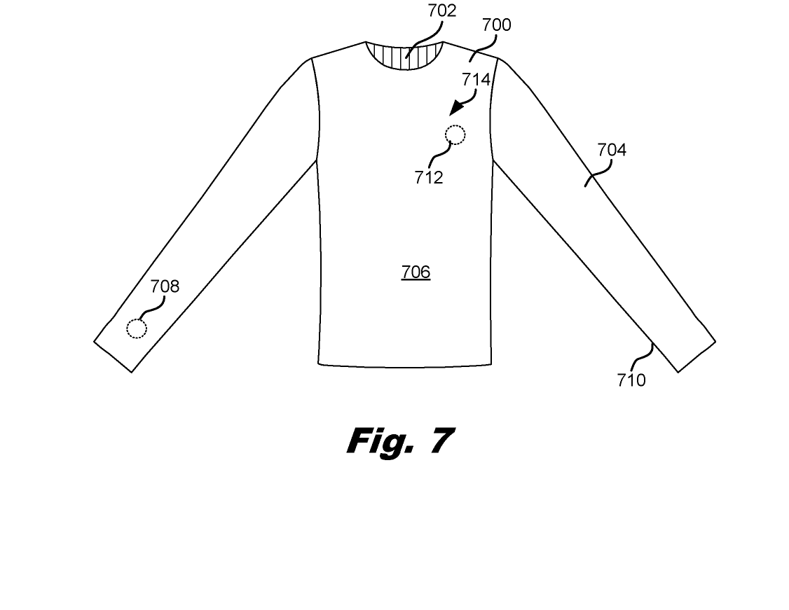

FIG. 7 depicts an example of an article 700 of clothing that can monitor a heart rate and/or apply a haptic input to influence the user's heart rate. In this example, the article of clothing includes a neck hole 702, arm sleeves 704, and a torso portion 706. An electrode 708 is incorporated into a wrist portion 710 of the sleeve 704 and can be used to monitor a user's heart rate. While the electrode is depicted in a specific location in the article of clothing, the electrode can be incorporated into the article of clothing in any appropriate location, such as a wrist portion, a back portion, a torso portion, a heart portion, a neck portion, a hand portion, an arm portion, a belly portion, another portion, or combinations thereof.

An haptic input 712 can be positioned in a heart portion 714 of the article of clothing to influence the user's heart rate. While the haptic input is depicted in a specific location in the article of clothing, the haptic input can be incorporated into the article of clothing in any appropriate location, such as a wrist portion, a back portion, a heart portion, a neck portion, a hand portion, an arm portion, torso portion, a belly portion, another portion, or combinations thereof. Further, any appropriate type of wearable device can include, but are not limited to, shirts, pants, shorts, dresses, socks, leggings, pajamas, sweaters, sweat shirts, scarfs, underwear, hats, gloves, mittens, thermals, jackets, tank tops, other types of clothing, or combinations thereof.

### General Description

When a healthy user lies down to sleep, the user can drift into the initial stages of sleep which are characterized by the user being in a semi-conscious state. As time progresses, the user falls into a deeper sleep. The first stages of sleep experienced by the user are referred to as non-rapid eye movement (non-REM) sleep. Often, during non-REM sleep, the user's body advances into a condition where the user's heart rate slows down, the user's breathing gets deeper and slower, and the user's muscles become more relaxed.

The final stage of sleep is rapid eye movement (REM) sleep where the user's brain activity and heart rate pick up again. During REM sleep, the user's eyes move side to side and the user can experience dreaming. REM sleep is the deepest sleep and generally, the user's muscles are often inhibited from moving during this stage of sleep. It can take a user between 90 and 120 minutes to advance through a single cycle of sleep. Upon completion of the first cycle, the user generally advances through the stages of the sleep cycle again. Often a user can complete four to six sleep cycles in a given night.

In general, the present disclosure can provide the user with system for assisting the user with sleeping and/or waking. Such a system can determine the user's natural or current heart rate with a heart rate monitor. The system can know or otherwise calculate a target heart rate to assist the user with sleeping and/or waking. Such a target heart rate can be used to assist the user with falling asleep, staying asleep, or waking from sleep. Haptic input that adjusts the user's heart to arrive at the target heart rate can be provided to the user. Such haptic input can have a beat that is at least similar to the target heart rate. In other examples, the haptic input is directed at slowly causing the user's heart rate to adjust to the target heart rate by using incremental beat rates in haptic input provided to the user for specific periods of time. The incremental beat rates can progressively adjust to the target heart rate.

The components of the adjustment system, such as a haptic device, interface, and heart rate monitor can be incorporated into a bed, articles of clothing, wearable devices, or combinations thereof. In some examples, the haptic device and/or the heart rate monitor are independent of the bed, but are in communication with the appropriate components of the adjustment system.

An adjustment system can be well suited for individuals who have sleeping disorders, especially those types of sleeping disorders that makes it difficult for the user to relax when trying to fall asleep. However, the adjustment system as described in the present disclosure can also be used to help the user stay asleep, or to wake from sleep. For example, by continuously providing haptic input to the user, the user's heart rate can stay at a desirable rate for sleeping. Further, as described herein, the system can be used to help the user progress through the various sleep stages and/or sleep cycles, and/or to progress to wakefulness. For example, the heart rate can be increased to help the user progress from non-REM sleep to REM sleep. Likewise, the heart rate can be adjusted to help the user move from REM sleep to non-REM sleep to help the user wake up. For example, if the adjustment system determines that the user is in REM sleep before the user's desired time period for waking, the adjustment system can provide haptic input to the user to cause the user to transition from REM sleep to non-REM sleep. Such a system can assist the user in waking up without feeling groggy.

While the examples above have been described with reference to an adjustment system that assists a single person with sleeping, the principles described herein can be applied to assisting multiple users sleep at once. For example, the system can include multiple users where each user is associated with a dedicated heart rate monitor and/or haptic device. In some cases, a single haptic device can be used to direct haptic input to both users simultaneously. In such an example, the haptic input can have a single beat that is customized to assist both users to fall asleep, stay asleep, and/or wake up. In other examples, one haptic device or independent haptic devices can direct focused haptic input to each user such that the haptic input affects the intended user without substantially affecting the unintended user. In this way, the haptic input directed to a user can be isolated from the other user. Such systems, with one or more haptic devices can be incorporated into a double bed, a queen sized bed, a king sized bed, a twin sized bed, a hammock, a fold out bed, another type of bed, or combinations thereof. In some cases, each user can wear a separate independent haptic device incorporated into articles of clothing, wearable devices, or combinations thereof.

In some embodiments where multiple users are assisted, the system can include programmed instructions to cause a processor to determine the natural heart rates of a one or more users, determine separate target heart rates for each user, and cause the one or more haptic devices to provide individualized and independent haptic input to each user to adjust the natural heart rate of each user to the target heart rate determined for that user.

In addition to determining the user's heart rate, the adjustment system 100 can also determine a target heart rate that can assist the user with sleeping. For example, the target heart rate, can be a heart rate that is associated with the user when the user enters into the early stages of sleeping. For example, non-REM stages of the sleep cycle are often characterized by a heart rate drop, and the target heart rate can be a heart rate exhibited by the user during such non-REM stages. In some cases, a user's target heart rate is about 6.0 to 10.0 percent lower than the user's resting heart rate. However, target heart rates can be affected by a host of factors including the user's age, weight, body composition, gender, overall fitness level, diet, other health factors, other factors, or combinations thereof.

In some embodiments, the heart rate monitor of an adjustment system can be a chest strap monitor, a wrist watch monitor, a monitor worn by the user, a monitor incorporated into the user's clothing, another type of heart rate monitor, or combinations thereof. Further, the heart rate monitor can detect electrical signals that are produced during the operation of a beating heart. Such electrical signals can be recorded by at least two electrodes in contact with the user's skin. However, other mechanisms for determining the user's heart rate can be used. For example, a microphone can be placed within a region where the microphone can pick up on the sounds made by the user's heartbeat. Further, the heart rate monitor can include a mechanism for detecting the user's pulse, and the heart rate monitor can determine the user's heart rate based on the pulse rate. While these examples have been described with reference to specific heart rate monitors, any appropriate mechanism for determining the user's heart rate can be used.

In some examples, a haptic device can produce a beat that is slower than the user's natural heart rate, but faster than the target heart rate. The haptic device can alternatively produce a beat that is faster than the user's natural heart rate, but slower than the target heart rate. Such intermediary heart rate haptic inputs can be used to slowly adjust the user's heart rate to the desired target heart rate. For example, the haptic input can mimic a beat that is 5.0 percent slower than the user's natural heart rate for a predetermined increment of time. During that increment of time, the user's heart rate can slow down to have the same rate as the beat of the haptic input. At the conclusion of the incremental time period, another slower beat can be caused to be emitted from the haptic device. As before, the user's heart rate can also slow down to mimic the rate of the subsequent haptic input. This process can repeat itself until the user's heart rate arrives at the target heart rate, where the target heart rate continues to be mimicked, or where the haptic input ceases.

In some cases, the haptic input can be provided until the user's heart rate arrives at the target heart rate, whereupon the haptic input ceases to be provided while the user's heart rate is within a predetermined amount of the target heart rate. For example, haptic input can be provided until the user's heart rate arrives at the target heart rate and then may not be further provided unless and until the user's heart rate is no longer within, for example, 2.0 percent, 5.0 percent, or 10 percent of the target rate. At that point, haptic input can again be provided until the user's heart rate arrives at the target heart rate, whereupon the haptic input ceases. This process can repeat itself until the user awakens. Thus, in some examples, the heart rate monitor 118 can continue determine if the natural heart rate is within a predetermined amount of the target heart rate even when haptic input is not being provided to the user.

The increments of time can be any appropriate length. For example, the time increments can be for 10.0 seconds, 20.0 seconds, 30.0 seconds, 1.0 minute, 2.0 minutes, another duration, or combinations thereof. Additionally, the increments of time can have different time lengths, which can depend on how much of a difference there is in the slower heart rate than the current heart rate of the user.

In some cases, the haptic input is provided by the haptic device just long enough for the user to establish a deep stage of sleep. In other cases, the haptic input is provided through just certain stages of sleep. For example, the haptic input can be directed to the user during just the non-REM stages of sleep or just certain stages of the non-REM sleep. Since the user's heart rate varies and often increases during REM sleep, the haptic input can be turned off during REM sleep to avoid influencing the user's heart rate during REM sleep. In yet other cases, the haptic input can be provided though all of the sleep cycle's stages, including during REM sleep. In other examples, the haptic input is provided at the conclusion of the user's REM sleep to assist the user in reentering the sleep cycle, or in entering a state of wakefulness, as can be determined by, for example, a preset time.

To determine the user's heart rate, the adjustment system can have access to profile information about the user in addition to any data provided by the heart rate monitor, such as the user's weight, body composition, height, age, gender, health conditions, other factors, or combinations thereof. Such profile information can be available to the adjustment system through an iFit program available through www.ifit.com and administered through ICON Health and Fitness, Inc. located in Logan, Utah, U.S.A. An example of a program that can be compatible with the principles described in this disclosure is described in U.S. Pat. No. 7,980,996 issued to Paul Hickman. U.S. Pat. No. 7,980,996 is herein incorporated by reference for all that it discloses. However, such profile information can be available through other types of programs that contain such information. For example, such information can be gleaned from social media websites, blogs, government databases, private databases, other sources, or combinations thereof. Also, the adjustment system can record the user's heart rate through the night through the heart rate monitor and send that information to the user's profile. Such information can allow the user to determine patterns about his or her sleep, become aware of sleeping conditions, track sleeping trends, perform other tasks, or combinations thereof. Further, the recorded information can be used by the adjustment system to learn which target heart rates were the most effective for helping the user sleep. For example, if the calculated target heart rate appears to be less effective than another heart rate, the system can adapt to the other heart rate. In such examples, the target heart rates can be customized for each individual.

The adjustment system can have a single target heart rate at which the adjustment system desires to impose on the user's heart rate. In such example, the user's heart rate can be brought to that rate, and the haptic input can cause the user's heart to maintain that rate. However, in other examples, the user's target heart rate can change over time. For example, the user's target heart rate can change depending on the stage of the user's sleep cycle. In some cases, the adjustment system can determine that the target heart rate for the user during the second stage of sleep is to be different than the target heart rate during the user's third stage of sleep. Further, the same stage in a sleep cycle can have different desirable heart rate depending on the number of sleep cycles that the user has already gone through that night. For example, during the initial sleep stages of the first cycle, the adjustment system can determine that the target heart rate is to have a first rate, while the target heart rate of the initial stages during the second sleep cycle is to have a second rate that is different than the first rate.

In some examples, the electrode and/or haptic input device are incorporated into an article of clothing such as a garment. The garment can include at least one sensor, such as a sensor that is incorporated into the garment's fabric. In some examples, a wireless device is incorporated into the garment's fabric which can send and receive signals from other sources. Such a wireless device can be in communication with a remote device that has information about the user's sleeping habits, sleep history, personal information, other types of information, or combinations thereof that can be useful in determining where the user's heart can be to induce sleep, induce a particular stage of sleep, wake the user, or combinations thereof. The remote device can be a mobile device, a laptop, a desktop, a cloud based device, a storage device, a digital device, another type of device, or combinations thereof.

In one example, the electrode or other type of sensor can be positioned adjacent regions of the user's body through the garment to receive electrical cardio signals of the user. Such electrical cardio signals can be used to determine the user's heart rate. The cardio signals can be a user's pulse, a blood flow signal, an optical signal, an electrical timing signal used by the user's body to control the heart rate, another type of signal, or combinations thereof.

In another example, an electrode can be positioned to receive electromyography signals that detect muscle contraction. This information can be used to determine how restful the user is sleeping. In those examples where the user is tossing and turning in the night, the muscle activity can be detected, which can be used by the monitoring system to determine where the heart rate can be at to assist the user with sleeping. The sensors and/or electrodes can be positioned over the user's deltoid muscles, bicep muscles, and forearm muscles. However, the surface electromyography sensors can be positioned proximate pectoral muscles, trapezius muscles, oblique muscles, abdominal muscles, latissimus dorsi muscles, tricep muscles, hamstring muscles, quadriceps muscles, calf muscles, adductor muscles, other types of muscles, or combinations thereof. As the muscles contract, the corresponding electromyography sensor can detect an electrical signal indicating the muscle contraction.

In some cases, the clothing can also include accelerometers. Such accelerometers can be incorporated into the garment in any appropriate location to determine the types of body movements performed by the user. For example, a three axis accelerometer can be incorporated into the garment to determine vertical and horizontal movements. The movement patterns can be analyzed to determine the user's types of movements. Accelerometers can also be used to determine a respiration count of the user. For example, at least one accelerometer positioned about the user's chest can be used to determine when the user's chest expands and contracts in accordance with the user's breathing. In other examples, a strain gauge can be incorporated into the garment, and as the user's chest expands from breathing, the strain gauge stretches. As the strain gauge stretches, it generates a signal that can be sent to the activity information device. The user's respiration data can also be used to determine how well the user is sleeping and whether a change should be made to the user's heart rate.

In some cases, a single sensor is used to determine the user's heart rate or other characteristic about the user's sleep. In other examples, multiple sensors can be incorporated into the bed, the article of clothing, a wearable device, or combinations thereof. Further, each of the sensors used to determine a characteristic about the user's sleep does not have to be of the same type of sensor. For example, a sensor can be dedicated to determining the user's pulse, another sensor for determining the respiration rate, another sensor for determining muscle contractions, another sensor to detected brain waves, another sensor detecting other types of sleep characteristics, or combination thereof.

In some cases, each sensor can be in communication with the wireless device. In some examples, each sensor is in communication with the wireless device through an independent electrically conductive medium. In other examples, the sensors communicate with each other and communicate with the wireless device through other sensors. For example, the wireless device can be in direct communication with a first sensor and indirectly in communication with a second sensor through the first sensor. The second sensor can send information towards the wireless device by sending the information to the first sensor, which then sends the information on to the wireless device. In such an example, the sensors form a network. Such a sensor network can allow sensors to communicate with each other. In some examples, such communications are bidirectional where the first sensor can send messages to the second sensor and the second sensor can send messages to the first sensor. Such networks can have any appropriate network topology, such as a daisy chain topology, a bus topology, a star topology, a mesh topology, ring topology, a tree topology, a linear topology, a fully connected topology, another type of topology, or combinations thereof.

An electrically conductive medium can include a cable or another type of wire that is disposed within channels formed within the fabric of the garment. In other examples, an electrically conductive thread is used to create an electrically conductive pathway formed in the fabric of the garment. For example, a single thread can be used to create the electrically conductive pathway. In other examples, multiple threads are used to form a patch of electrically conductive fabric capable of conducting an electrical signal. Such an electrically conductive fabric can be covered by an outer fabric layer, an inner fabric layer, a waterproof layer, a breathable layer, another type of layer, or combinations thereof. In some examples, an electrically conductive fabric is exposed in the inner or outer surfaces of the garment. In some cases, the electrodes or other types of sensors in the clothing or other type of wearable device can be held in compression against the user's body. This can be accomplished through the user of elastic material incorporated into the clothing.

While the above examples have been described with the sensors being in communication with each other or with the wireless device, any appropriate communication mechanisms can be used to enable communication between the components of the garment. For example, the sensors can be in communication with each other through fiber optic cables, wireless transceivers, other types of communication channels, or combinations thereof. In some examples, the garment includes multiple wireless devices that are capable of communication with the activity information device directly or indirectly.

Further, while the above examples have been described with reference to performing calculations and other forms of interpreting the data collected by the sensors with the activity information device, any appropriate location for performing such calculations and/or interpretations can be used in accordance with the principles described in the present disclosure. For example, such processing can occur on the mobile device, a networked device, a computing device incorporated into the garment, another type of device, or combinations thereof.

In some examples, a battery or another type of power source is incorporated into the garment and/or a wearable device. The battery can be a disposable battery or a rechargeable battery. In some cases, the garment can include an energy harvesting mechanism, such as a linear generator that can harvest the movements of the user to produce energy or a thermoelectric device that can use the thermal differential between the user's body heat and the ambient temperature of the air surrounding the user to provide energy to power the sensors of the garment. In some examples, such energy harvesting mechanisms supplement the battery or other power source in the garment or such an energy harvesting mechanism can be used to recharge such batteries.

What is claimed is:

1. A system for adjusting a heart rate, comprising:
a heart rate monitor;
a haptic device; and
a processor and memory, the memory including programmed instructions to cause the processor to:
determine a natural heart rate of a user;
determine a target heart rate for the user; and
cause the haptic device to provide a haptic input to the user to adjust the natural heart rate to the target heart rate.

2. The system of claim 1, further comprising an interface in communication with the heart rate monitor and the haptic device.

3. The system of claim 2, wherein the haptic device is communicatively coupled to the processor.

4. The system of claim 3, wherein the haptic device comprises a cone-less speaker.

5. The system of claim 3, wherein the haptic device comprises an electromagnetic transducer.

6. The system of claim 3, wherein the haptic device is incorporated into a bed, an article of clothing, a wearable device, or combinations thereof.

7. The system of claim 1, wherein the target heart rate is configured to assist the user with waking out of a slumber.

8. The system of claim 7, wherein the target heart rate is higher than the natural heart rate.

9. The system of claim 1, wherein the haptic input is adjusted over a time period; and
wherein the haptic input at a beginning of the time period is closer to the natural heart rate and the haptic input at an end of the time period is closer to the target heart rate.

10. The system of claim 9, wherein the haptic input is changed incrementally during the time period.

11. The system of claim 9, wherein the time period ends when the natural heart rate reaches the target heart rate; and
wherein the programmed instructions cause the haptic device to cease providing haptic input while the natural heart rate is within a predetermined amount of the target heart rate.

12. The system of claim 1, wherein the programmed instructions further cause the processor to:
determine if the natural heart rate is within a predetermined amount of the target heart rate;
cause the haptic device to cease providing haptic input when the natural heart rate is within the predetermined amount of the target heart rate; and
cause the haptic device to provide the haptic input to the user to adjust the natural heart rate to the target heart rate when the natural heart rate is outside the predetermined amount of the target heart rate.

13. The system of claim 1, further comprising:
a second heart rate monitor;
wherein the programmed instructions further cause the processor to:
determine the natural heart rate of a second user;
determine the target heart rate of the second user; and
cause the haptic device to provide a second haptic input to the second user to adjust the natural heart rate to the target heart rate of the second user;
wherein the haptic input and the second haptic input are provided independently from one another.

14. A system for adjusting a heart rate, comprising:
a communication interface;
a heart monitor in communication with the communication interface;
a haptic device in communication with the communication interface; and
a processor and memory, the memory including programmed instructions to cause the processor to:
determine a natural heart rate of a user;
determine a target heart rate configured to assist the user with sleeping; and
cause the haptic device to provide a haptic input to the user to adjust the natural heart rate to the target heart rate where the haptic input is adjusted over a time period to where the haptic input at a beginning of the time period is closer to the natural heart rate and the haptic input at an end of the time period is closer to the target heart rate.

15. The system of claim 14, wherein the haptic input is changed incrementally during the time period.

16. The system of claim 14, wherein the heart monitor is worn on a body of the user.

17. The system of claim 14, wherein the haptic device is incorporated into a bed, an article of clothing, or a wearable device.

18. The system of claim 14, wherein the heart monitor is in wireless communication with the processor.

19. The system of claim 14, wherein the target heart rate is determined based on a desired state of a sleep or wakefulness of the user.

20. A system for adjusting a heart rate, comprising:
a communication interface;
a heart monitor worn by a user and in communication with the communication interface;
a haptic device incorporated into a bed, an article of clothing, or a wearable device and in communication with the communication interface; and
a processor and memory, the memory including programmed instructions to cause the processor to:
determine a natural heart rate of a user;
determine a target heart rate configured to assist the user with sleeping or waking;
cause the haptic device to provide a haptic input to adjust the natural heart rate to the target heart rate where the haptic input is adjusted incrementally over a time period to where that the haptic input at a beginning of the time period is closer to the natural heart rate and the haptic input at an end of the time period is closer to the target heart rate; and
cause the haptic device to cease providing haptic input when the natural heart rate is within a predetermined amount of the target heart rate.
